(19) United States
(12) Patent Application Publication
(10) Pub. No.: US 2023/0013453 A1
(43) Pub. Date: Jan. 19, 2023

(54) RESVERATROL SKIN PREPARATION

(71) Applicant: Matthew D Edwards, Dallas, TX (US)

(72) Inventor: Matthew D Edwards, Dallas, TX (US)

(21) Appl. No.: 17/866,393

(22) Filed: Jul. 15, 2022

Related U.S. Application Data
(60) Provisional application No. 63/222,262, filed on Jul. 15, 2021.

(51) Int. Cl. A61K 36/36

(52) U.S. Cl. CPC A61K 36/36

(57) ABSTRACT

The present invention discloses a method for using sonication to achieve a homogenized blended oil and trans-resveratrol base and functions as a multi-use men's skin. hair, and anti-aging product for irritated skin. The Trans-resveratrol used in the present disclosure is sourced from Japanese Knotweed, Polygonum cuspidatum roots. Trans-resveratrol is a super antioxidant with a high concentration of polyphenols. Trans-resveratrol is derived from the Japanese Knotweed plant and is significantly more potent than the more commonly known, cis-resveratrol. The healing properties of Trans-resveratrol include decreased inflammation, improved cardiovascular health, and anti-aging effects, reduced impact of hangovers and intoxication, increased antioxidants in a person's blood stream, improved the appearance of complexion, reduced impact on skin from exhaustion or lack of sleep, healed body and muscles after exercise, and reduced impact of stress on body.

### PRIORITY

This nonprovisional utility patent applications claims priority to the provisional utility patent (Application No. 63/222,262) filed on Jul. 15, 2021.

### FIELD OF THE INVENTION

The present disclosure relates to an organic, topical skin preparation formulated with trans-resveratrol that is oil soluble, stable and bioavailable with quick absorbability.

### BACKGROUND

People have used topical creams, preparations and ointments for decades in an effort to protect their skin from the elements and irritants as well as to preserve or restore the skin's youthful appearance and smooth texture. The present compositions are formulated with both natural and synthetic ingredients. Generally, less expensive products have less pure or natural ingredients and typically have a longer shelf-life due preservatives and other chemicals. While organic and natural products typically have a shorter shelf-life due to their lack of preservatives and stabilizing chemicals. These skin preparations may be either water based or oil based.

Resveratrol consists of two aromatic rings attached by a methylene bridge. Resveratrol is a polyphenol compound found in certain plants and in red wine that has antioxidant properties and has been possible anticarcinogenic effects. It is a natural phenol and phytoalexin which is produced naturally by different plant species such as grapevines and legumes. Resveratrol is a multipurpose substance that improves health and both prevents and treats chronic disease. One of the well-known, often used ingredients for skin treatments is cis-resveratrol, which is found in grapes, wine, peanuts and cocoa. Both cis-resveratrol and trans-resveratrol may be glycosylated but only the trans-resveratrol version is biologically active. Cis-resveratrol has less bioavailability due to its larger molecular size and is likely to become denatured at high temperatures and during processing, which decreases its effectiveness and shelf life. By contrast, Trans-resveratrol has a smaller molecular size and remains stable at high temperatures during processing. Trans-resveratrol is a super antioxidant with a high concentration of polyphenols. Trans-resveratrol is derived from the Japanese Knotweed plant and is significantly more potent than the more commonly known, cis-resveratrol. The healing properties of Trans-resveratrol include decreased inflammation, improved cardiovascular health, and anti-aging effects. When protected from light sources, trans-resveratrol is more potent, more bioactive, more effective and more stable than cis-resveratrol. Trans-resveratrol has not typically been used with an oil based because the trans-resveratrol does not break-down in oil. The present method uses sonication to achieve a homogenized blended oil and trans-resveratrol base to achieve the desired result on a large scale for manufacturing purposes. Sonication, a cell disruption method which uses high frequency sound waves to break cells, at approximately 26.000 pulses per second achieves the desired results for the trans-resveratrol skin preparation. Without sonication, the expense of combining trans-resveratrol and oil would be cost prohibitive at the large scale required for commercial manufacturing.

The present disclosure functions as a multi-use men's skin, hair, and anti-aging product for irritated skin and scalp. But it may also be used for women and children who desire organic skin products. The present invention has unexpected effects and is a multi-purpose skin preparation that alleviates eczema (dermatitis), itchy irritation of the dry or scaly skin while also hydrating the scalp. The Trans-resveratrol used in the present disclosure is sourced from Japanese Knotweed. Polygonum cuspidatum, roots, which is a member of the buckwheat family. After the Japanese Knotwood plant is farmed and harvested, the roots are isolated and the trans-resveratrol is extracted. The final product is almost completely pure with 98-99% pure trans-resveratrol with as much as 495 milligrams of trans-resveratrol per capsule in certain embodiments wherein a single capsule of the ME product contains the trans-resveratrol equivalent of 450 bottles of wine.

### OBJECTIVES

An object of one embodiment of the present disclosure is the sonication of trans-resveratrol in oil to create a superior skin preparation substance.

Another object of the present disclosure is to provide a method to enhance and protect user's skin through improved absorption of unadulterated trans-resveratrol.

Another object of the present disclosure is to provide a skin preparation that lowers inflammation, reduces oxidative stress, and offsets the effect of aging.

Another object of the present disclosure is to provide a skin preparation that accelerates recovery from heavy alcohol consumption and also improves a person's complexion after consumption of alcohol.

Another object of the present disclosure is to provide a skin preparation that increases antioxidants in a user's blood.

Another object of the present disclosure is to provide relief to sufferers of atopic dermatitis or eczema.

Another object of the present disclosure is to provide a packaging method to preserve the skin preparation without synthetic ingredients, additives and preservatives.

### SUMMARY

The present invention is a trans-resveratrol-based skin preparation, which is a pure, organic, anti-aging antioxidant skin and hair serum with trans-resveratrol concentrate extracted from pure 99% grade Japanese knotweed. This trans-resveratrol-based skin preparation is uniquely created for bioavailability with rapid skin absorption and eczema reduction effects when applied topically. The present invention relates to an oil soluble trans-resveratrol-based skin preparation or composition that may contain: 50% organic capric triglyceride, 20% organic jojoba oil, 25% organic rosehip oil and 5% trans-resveratrol in one embodiment but these percentages may be altered to address different needs of the end consumer. The trans-resveratrol-based skin preparation may also be used as a skin moisturizer and treatment for other common skin conditions.

Trans-resveratrol, also known as Polygonum cuspidatum, is a super antioxidant with a high concentration of polyphenols. Japanese knotwood has considerably more concentrated trans-resveratrol than red wine or grape juice and is often used in medical research. The present invention has many benefits over existing skincare formulations, for instance, it reduces inflammation, reduces impact of hangovers and intoxication, increases antioxidants in a person's blood stream, improves the appearance of complexion, reduces impact on skin from exhaustion or lack of sleep, heals body and muscles after exercise, and reduces impact of stress on body.

The present method of preparing this trans-resveratrol-based skin preparation uses sonication to achieve a homogenized blend of oil and trans-resveratrol base made from a trans-resveratrol concentrate. The Trans-resveratrol concentrate is produced from by isolating trans-resveratrol from the roots of Japanese Knotwood plants.

Sonication, a cell disruption method which uses high frequency sound waves to break cells, at 26,000 pulses per second achieves the desired results for the trans-resveratrol skin preparation. Without sonication, the expense of combining trans-resveratrol and oil would be cost prohibitive at the large scale required for commercial manufacturing of this trans-resveratrol skin preparation. The present formulation typically contains unique ingredients such as organic capric triglyceride, organic jojoba oil, organic rosehip oil and trans-resveratrol. By contrast, most products from competitors typically contain dozens of ingredients, including additional additives, including some which may have been linked to cancer, to contribute to the long shelf life of those competitor products. The present disclosure is devoid of such synthetic substances, adulterants and additives, which enhances the natural qualities and features of the ingredients of the trans-resveratrol skin preparation. Trans-resveratrol is more stable than cis-resveratrol and can be stable for over forty hours when protected from light and is more effective than the cis-resveratrol compound.

The present disclosure of this trans-resveratrol-based skin preparation is 99% pure grade Japanese knotweed trans-resveratrol instead of the less expensive counterpart, cis-resveratrol. Trans-resveratrol is oil soluble and bioavailable and can enter circulation when introduced into the body and has a resulting active effect. During manufacture, the trans-resveratrol-based skin preparation is heated precisely at 100° F. which is a low enough heat to preserve the integrity of trans-resveratrol while allowing for sufficient sonication and homogenization. The trans-resveratrol-based skin preparation manufacturing process heats the trans-resveratrol concentrate without excessive exposure to light, which could denature the antioxidant features of the trans-resveratrol concentrate and significantly decrease bioavailability of the final trans-resveratrol-based skin preparation. The trans-resveratrol-based skin preparation is then homogenized at precisely 30,000 RPM with select oils to achieve oil fixability, smooth viscosity, and pH that results in quick absorbability of the trans-resveratrol-based skin preparation. The level of high sheer through the turning stabilization process in the homogenizer creates suction with a smaller amount of heat and less exposure to light to keep the trans-resveratrol-based skin preparation's antioxidants effective and stable. The trans-resveratrol-based skin preparation manufacturing process also implements Ostwald ripening, the eventual change of an inhomogeneous structure, for segregation of the trans-resveratrol-based skin preparation overtone at homogenization to create stabilization without the addition of paraben agents and preserving the organic, natural condition of the ME product, its effectiveness and longer shelf life.

The antioxidant essential moisturizer product of trans-resveratrol-based skin preparation has increased oxidation through machine homogenization with an Ostwald ripening segregation after stabilization of trans-resveratrol. The trans-resveratrol-based skin preparation is then further broken down to a smaller molecular size so the antioxidant of the trans-resveratrol-based skin preparation can be absorbed into the skin since the molecules are now at 0.2% the size of the original size. This particularly small molecule size is an effective treatment for eczema since the antioxidant can be absorbed into the skin in its smallest molecular form. Other technical features may be readily apparent to those skilled in the art from the following figures and descriptions.

### BRIEF DESCRIPTION OF THE DRAWINGS

For a more complete understanding of this disclosure and its features, reference is now made to the following description, taken in conjunction with the accompanying drawings, in which:

FIG. 1 Japanese Knotwood or Polygonum cuspidatum plants.

FIG. 2 shows the harvesting process for resveratrol.

FIG. 3 shows the sonication process for resveratrol.

FIG. 4 shows the molecular structure of resveratrol.

FIG. 5 shows the detailed formulation of trans-resveratrol.

### DETAILED DESCRIPTION OF DRAWINGS

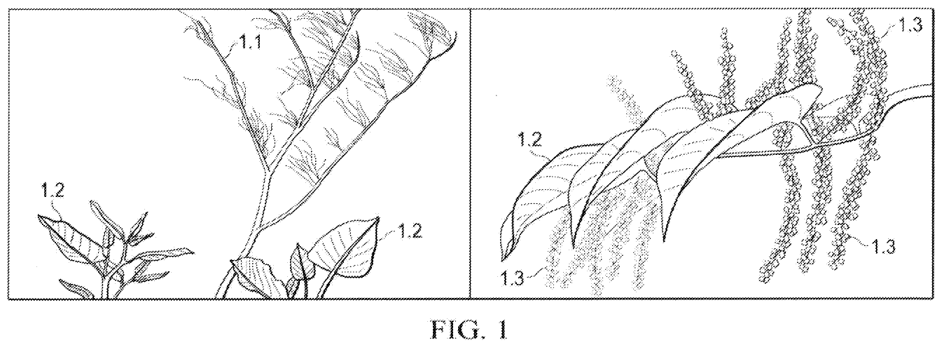

The present disclosure generally provides a method of manufacture fir a trans-resveratrol skin preparation. FIG. 1 shows the Japanese Knotwood or Polygonum cuspidatum plants. Polygonum cuspidatum is a member of the buckwheat family and the plant is upright, shrubby, herbaceous. The plant is a woody perennial that can grow to heights of ten to fifteen feet. The stems 1.1 are smooth, stout, and hollow. The plant bears large, heart shaped leaves 1.2 and has branched sprays of small white flowers 1.3, which bloom in late summer and fall. The Japanese knotwood plant also develops small triangular winged seeds. The roots of the Japanese Knotwood plant are processed to extract trans-resveratrol.

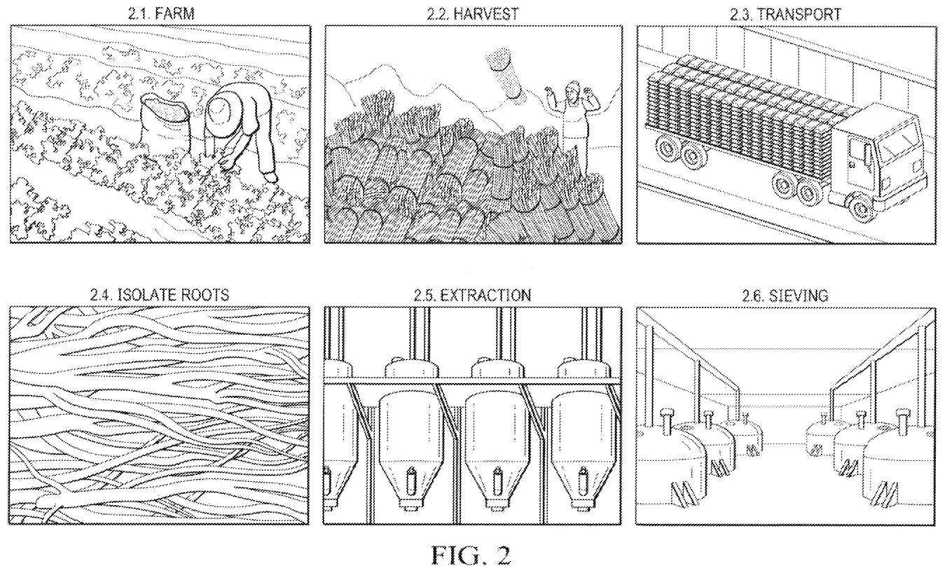

FIG. 2 shows the production and harvesting process for resveratrol. Crops that contain trans-resveratrol, such as certain fruits and vegetables as well as Japanese Knotwood plants are grown on farms 2.1. The crops or Japanese knotwood plants are harvested 2.2 and transported 2.3 to processing facilities. At these facilities, the roots of the Japanese knotwood plants are isolated 2.4 from the other portions of the plant. The Japanese knotwood roots are heated and the trans-resveratrol concentrate is extracted 2.5 from the plant's roots. The extracted trans-resveratrol concentrate solution is then sieved 2.6 to isolate the trans-resveratrol from the plant pans. The trans-resveratrol concentrate may be stabilized and combined with the other ingredients, such as oil and/or triglycerides, to produce the trans-resveratrol-based skin preparation.

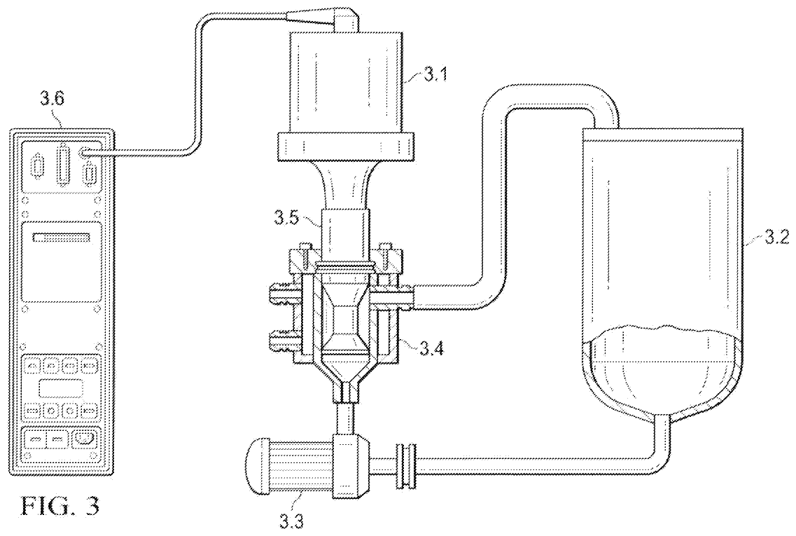

FIG. 3 shows stabilization of trans-resveratrol in an oil form using a Q-Sonica ultrasonic machine running at 24 hz. The extracted and sieved trans-resveratrol concentrate passes through an additional chilling unit, water-cooled transducer 3.1, as the jacketed tank, reactor chamber with jacket 3.3, cannot maintain the batch of trans-resveratrol at a low enough temperature for optimal cell disruption. All ingredients of the present disclosure are added into the storage tank 3.2 and mixed for approximately sixty (60) minutes before the pump 3.4 is turned on to shuttle the sample towards the half-wave barbell horn 3.5 of the sonication probe. The barbell horn amplifies the ultrasonic amplitudes while maintaining large output tip diameters, which makes it possible to scale up high intensity ultrasonic processes such as the present method of preparing trans-resveratrol skin preparation. This cycle consists of a ninety (90) second sonication phase and ninety (90) second pause, repeated for a total of thirty (30) minutes of sonication time. This results in a one (1) hour total run time with the pump 3.4 on and two (2) hours total mixing time for both the “Non-Pump” and “Pump” phasing, this sonication system and process is powered by a generator with amplitude, frequency and power display 3.6.

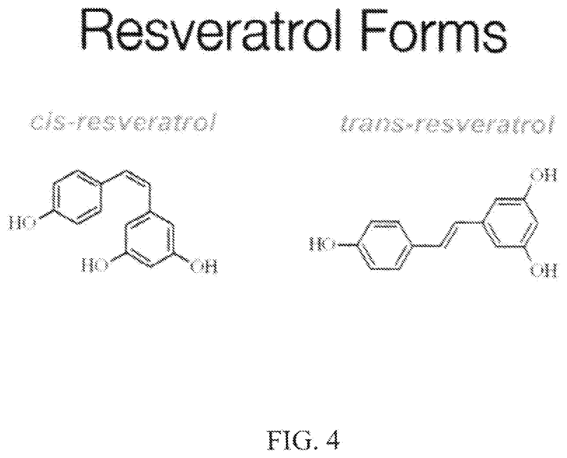

FIG. 4 shows the molecular structure of resveratrol, cis resveratrol versus trans-resveratrol. The trans-resveratrol molecule is a super antioxidant which is biologically active and the preferred substance for the present trans-resveratrol skin preparation. Trans-resveratrol is generally less widely known but is more potent than the cis-resveratrol. Trans-resveratrol is also more stable than Cis-resveratrol when protected from light. The healing properties of resveratrol include decreased inflammation, improved cardiovascular health, and anti-aging effects on the skin and body. Once the present formula is manufactured, it must be protected from light in order to prevent oxidation and decreased shelf-life. The present formulation is most optimally stored in dark bottles made of glass or stainless steel. The present formulation contains unique ingredients such as organic capric triglyceride, organic jojoba oil, organic rosehip oil and trans-resveratrol. Products from competitors typically contain possibly dozens of ingredients, including additional additives, including some which may have been linked to cancer, and also contribute to the long shelf life of those competitor products. The present disclosure is devoid of such adulterants and additives, which impact the natural properties of the skin preparation.

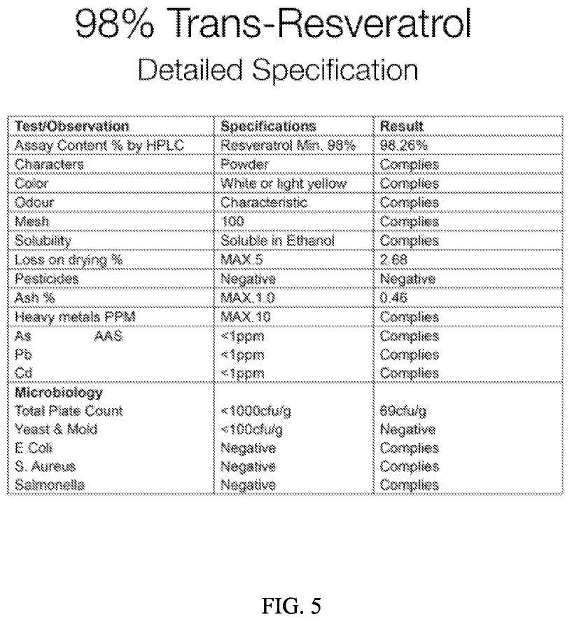

FIG. 5 shows the detailed specification of 98% trans-resveratrol including test/observation, specifications, and results. The extracted or sieved trans-resveratrol must be 98% trans-resveratrol at a minimum. It may be in powder form and either white or light yellow in color. It is also soluble in ethanol and does not contain pesticides. It is also devoid of any significant amounts of metals, bacteria or other adulterants or additives.

What is claimed is:

1. A method for the production of a trans-resveratrol-based skin preparation comprised of:
triglyceride, oils, and trans-resveratrol concentrate.

2. The method of claim 1 wherein said trans-resveratrol concentrate is sourced from Japanese Knotwood plants; wherein said Japanese Knotwood plants are further comprised of stems, leaves, flowers and roots.

3. The method of claim 2, wherein the production of said trans-resveratrol concentrate consists of:
Farming Japanese Knotwood plants;
Harvesting roots of said Japanese Knotwood plants:
Transporting said Japanese Knotwood roots:
Isolating said Japanese Knotwood roots;
Extraction trans-resveratrol from said Japanese Knotwood roots:
Sieving trans-resveratrol concentrate from said extraction of trans-resveratrol from said Japanese Roots.

4. The method of claim 3 wherein the production of said trans-resveratrol-based skin preparation consists of:
Passing trans-resveratrol concentrate through a water-cooled transducer;
Passing trans-resveratrol concentrate through a jacketed reactor chamber:
Mixing trans-resveratrol concentrate with oil product to create trans-resveratrol oil product inside storage tank;
Pumping trans-resveratrol oil product to a sonication probe to produce trans-resveratrol based skin preparation;
Storing said trans-resveratrol-based skin preparation in a light resistant, stable container.

5. The method of claim 4, wherein the production of a skin preparation further utilizes Ostwald ripening to further stabilize said skin preparation.

6. A skin preparation comprised of capric triglyceride, jojoba oil, rosehip oil and trans-resveratrol.

7. A method of treating skin conditions wherein a trans-resveratrol-based skin preparation is created from triglycerides, oils and trans-resveratrol, wherein said skin preparation is organic, wherein said skin preparation is natural, wherein said skin preparation is shelf-stable.

8. The method of claim 7 wherein said trans-resveratrol-based skin preparation is produced using sonication and Ostwald ripening to provide stabilization.
